(19) United States
(12) Patent Application Publication
(10) Pub. No.: US 2024/0268978 A1
(43) Pub. Date: Aug. 15, 2024

(54) COMMISSURAL ALIGNMENT OF TRANSCATHETER HEART VALVE DURING TRANSCATHETER AORTIC VALVE REPLACEMENT

(71) Applicant: Cedars-Sinai Medical Center, Los Angeles, CA (US)

(72) Inventors: Tarun Chakravarty, Sherman Oaks, CA (US); Raj Makkar, Beverly Hills, CA (US); Vivek Patel, Northridge, CA (US)

(73) Assignee: Cedars-Sinai Medical Center, Los Angeles, CA (US)

(21) Appl. No.: 18/568,184

(22) PCT Filed: Jun. 8, 2022
(86) PCT No.: PCT/US2022/032641

§ 371 Date: Dec. 7, 2023

Related U.S. Application Data
(60) Provisional application No. 63/208,672, filed on Jun. 9, 2021.

(51) Int. Cl. A61F 2/95; A61F 2/24

(52) U.S. Cl. CPC A61F 2/9524; A61F 2/2412; A61F 2/2433

(57) ABSTRACT

Devices, systems and methods are provided for achieving commissural alignment when a transcatheter heart valve (THV) is implanted during a transcatheter heart valve replacement (THVR) procedure. For instance, a method in accordance with one approach is for preparing a prosthetic heart valve for a transcatheter heart valve procedure. The method includes: determining a native heart valve commissural orientation according to one or more images acquired via a cardiac imaging modality. In response to the determination, the prosthetic heart valve is further crimped according to the native heart valve orientation. In one example, these devices, systems and methods may be used to diagnose and/or treat patients with a valvular heart disease, particularly those who have aortic stenosis or regurgitation. Further, in some examples, the devices, systems and methods described herein are implemented for achieving commissural alignment when a balloon expandable THV is used for TAVR.

### CROSS-REFERENCE TO RELATED APPLICATIONS

This application claims priority from and benefit of U.S. Provisional Patent Application Ser. No. 63/208,672, filed Jun. 9, 2021, titled “COMMISSURAL ALIGNMENT OF TRANSCATHETER HEART VALVE DURING TRANSCATHETER AORTIC VALVE REPLACEMENT,” which is hereby incorporated by reference herein in its entirety.

### FIELD OF INVENTION

This invention relates to devices, systems and methods for transcatheter heart valve replacement; and more particularly to transcatheter aortic valve replacement (TAVR).

### BACKGROUND OF THE INVENTION

The following description includes information that may be useful in understanding the present invention. It is not an admission that any of the information provided herein is prior art or relevant to the presently claimed invention, or that any publication specifically or implicitly referenced is prior art.

Transcatheter aortic valve replacement (TAVR, also known as transcatheter aortic valve implantation (TAVI)) is a procedure for patients with severe symptomatic aortic stenosis (narrowing of the aortic valve opening). During TAVR, a transcatheter heart valve (THV) is inserted percutaneously using a catheter and implanted in an orifice of a native aortic valve. Since TAVR is not performed under direct visualization of the native aortic valve (unlike open heart surgery), alignment of the THV with the native aortic valve is rarely achieved.

Current TAVR procedures suffer from mis-alignment of THV commissures with native aortic valve commissures. Commissure misalignment may impede coronary access for selective angiography and percutaneous coronary intervention (PCI) due to position of the commissural suture posts, transcatheter heart valve leaflets or the external skirt of the transcatheter heart valve in relation to the coronary artery (Ochiai T. et al. JACC: Cardiovascular Interventions 2020. PMID 32192689). Further, rates of paravalvular regurgitation (Fuchs A. et al. JACC: Cardiovascular Interventions 2018. PMID 30121280) and incidence of prosthesis-patient mismatch has been reported to be higher in patients with THV commissural misalignment compared with patients who have commissural alignment. Furthermore, commissural alignment is important to preserve coronary access after TAVR-in-TAVR. Further still, computational modeling has suggested that commissural alignment may reduce THV leaflet stress, particularly in the setting of elliptical annuli (Gunning et al. Ann Biomed Eng. 2014. PMID: 24912765). Thus, there is a need for achieving commissural alignment when TAVR is performed.

However, alignment of the THV commissures with the native aortic valve commissures (THV commissures within 0°-15° of the native aortic valve commissures) is achieved in only ˜25% of the patients undergoing TAVR. Up to 50% of patients undergoing TAVR have moderate misalignment (30°-45°) or severe misalignment (450-60°) of the THV commissures compared with the native aortic valve commissures, and about 25% of the patients suffer from mild misalignment (150-30°).

An example approach to obtain commissural alignment between THV and native aortic valves is shown by Tang G. et al. (JACC Cardiovasc Interv. 2020 May 11; 13(9):1030-1042. PMID: 32192985). Therein, one of the commissural posts of a balloon-expandable valve is crimped at an initial orientation, such as 3, 6, 9, or 12 o'clock. However, with such an approach, Tang et al., were not able to achieve commissural alignment. It follows that, conventionally, there has been no solution to achieve commissural alignment between the THV and native aortic valve.

### SUMMARY

The inventors herein have identified many disadvantages with the above-mentioned approach. As an example, the crimping of the balloon-expandable valve is performed using a random angle without considering a native aortic valve anatomy. As a result, desired alignment between the balloon-expandable valve and the native aortic valve is not achieved.

In order to at least partially address the above-mentioned issues, the inventors herein provide systems and methods for achieving commissural alignment between THV and native heart valve in patients undergoing transcatheter valve replacement procedures. In one example, a method for preparing a prosthetic heart valve comprises: determining a native heart valve orientation according to one or more images acquired via a cardiac imaging modality; and crimping the prosthetic heart valve according to the native heart valve orientation.

In this way, by determining a native heart valve orientation prior and crimping a prosthetic heart valve based on the native heart valve orientation, improved commissural alignment is achieved. This desirably facilitates commissural alignment after TAVR, especially with the balloon-expandable THVs. Modification in THV device technology, design, valve crimping or delivery methods, based on the approaches described herein, has the potential to improve commissural alignment and widespread adoption.

For instance, a method in accordance with one approach is for preparing a prosthetic heart valve for a transcatheter heart valve procedure. The method includes: determining a native heart valve commissural orientation according to one or more images acquired via a cardiac imaging modality. In response to the determination, the prosthetic heart valve is further crimped according to the native heart valve orientation.

A method in accordance with another approach is for crimping a prosthetic heart valve. Here, the method includes: determining a crimping orientation according to a native heart valve orientation. The prosthetic heart valve is further positioned in a crimping aperture of a crimping device at the crimping orientation, and a crimping lever is actuated to actually crimp the prosthetic valve.

A method according to yet another approach is for crimping a prosthetic heart valve. The method includes: determining at least one native commissure orientation of a native heart valve, and crimping the prosthetic heart valve according to the native commissure orientation.

A device according to another approach is for crimping a prosthetic heart valve. The device includes: an external housing including a crimping aperture, and a circular scale disposed around a circumference of the crimping aperture. The circular scale includes radially oriented crimping angles, and the radially oriented crimping angles correlate with one or more native heart commissure angles.

The advantages introduced above, and other advantages/features of the present description, will be readily apparent from the following Detailed Description when taken alone or in connection with the accompanying drawings. It should be understood that the summary above is provided to introduce in simplified form a selection of concepts that are further described in the detailed description. It is not meant to identify key or essential features of the claimed subject matter, the scope of which is defined uniquely by the claims that follow the detailed description. Furthermore, the claimed subject matter is not limited to implementations that solve any disadvantages noted above or in any part of this disclosure.

The above summary is not intended to represent each embodiment or every aspect of the present disclosure. Rather, the foregoing summary merely provides an example of some of the novel aspects and features set forth herein. The above features and advantages, and other features and advantages of the present disclosure, will be readily apparent from the following detailed description of representative approaches and modes for carrying out the present invention, when taken in connection with the accompanying drawings and the appended claims. Additional aspects of the disclosure will be apparent to those of ordinary skill in the art in view of the detailed description of various approaches, which is made with reference to the drawings, a brief description of which is provided below.

### BRIEF DESCRIPTION OF DRAWINGS

The accompanying drawings, which are incorporated in and constitute a part of this specification, exemplify the embodiments of the present invention and, together with the description, serve to explain and illustrate principles of the invention. The drawings are intended to illustrate major features of the exemplary embodiments in a diagrammatic manner. The drawings are not intended to depict every feature of actual embodiments nor relative dimensions of the depicted elements, and are not drawn to scale.

FIG. 1A is a block diagram illustrating a transcatheter heart valve system, according to an embodiment of the disclosure.

FIG. 1B is a flow chart illustrating a high-level method for preparing a transcatheter heart valve for a transcatheter heart valve replacement (THVR) procedure, according to an embodiment of the disclosure.

FIG. 2 is an example computed tomography image for determining a native heart valve orientation, according to an embodiment of the disclosure.

FIG. 3 is a schematic illustration of an example balloon-inflatable heart valve at a crimping orientation based on the native heart valve orientation shown at FIG. 2, according to an embodiment of the disclosure.

FIG. 4 is a schematic illustration of an example crimper including indications corresponding to native heart valve orientations, according to an embodiment of the disclosure.

FIG. 5 is a schematic illustration of an example transcatheter heart valve delivery system, according to an embodiment of the disclosure.

FIG. 6 is an image of a cross-section of a heart depicting commissural alignment of a transcatheter heart valve with respect to a native heart valve after a THVR procedure.

### DETAILED DESCRIPTION OF THE INVENTION

Unless defined otherwise, technical and scientific terms used herein have the same meaning as commonly understood by one of ordinary skill in the art to which this invention belongs. Szycher's Dictionary of Medical Devices CRC Press, 1995, may provide useful guidance to many of the terms and phrases used herein. One skilled in the art will recognize many methods and materials similar or equivalent to those described herein, which could be used in the practice of the present invention. Indeed, the present invention is in no way limited to the methods and materials specifically described.

In some embodiments, properties such as dimensions, shapes, relative positions, and so forth, used to describe and claim certain embodiments of the invention are to be understood as being modified by the term “about.”

Various examples of the invention will now be described. The following description provides specific details for a thorough understanding and enabling description of these examples. One skilled in the relevant art will understand, however, that the invention may be practiced without many of these details. Likewise, one skilled in the relevant art will also understand that the invention can include many other obvious features not described in detail herein. Additionally, some well-known structures or functions may not be shown or described in detail below, so as to avoid unnecessarily obscuring the relevant description.

The terminology used below is to be interpreted in its broadest reasonable manner, even though it is being used in conjunction with a detailed description of certain specific examples of the invention. Indeed, certain terms may even be emphasized below; however, any terminology intended to be interpreted in any restricted manner will be overtly and specifically defined as such in this Detailed Description section.

While this specification contains many specific implementation details, these should not be construed as limitations on the scope of any inventions or of what may be claimed, but rather as descriptions of features specific to particular implementations of particular inventions. Certain features that are described in this specification in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations may be depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the implementations described above should not be understood as requiring such separation in all implementations, and it should be understood that the described program components and systems can generally be integrated together in a single software product or packaged into multiple software products.

One skilled in the art will recognize many methods and materials similar or equivalent to those described herein, which could be used in the practice of the present invention. Indeed, the present invention is in no way limited to the methods and materials described. For purposes of the present invention, the following terms are defined below.

In various approaches, the devices, systems and methods described herein are configured for humans. One of skill in the art would readily appreciate that the devices, systems and methods described herein could be customized for use in almost any mammal in which a heart valve may be replaced.

“Mammal” as used herein refers to any member of the class Mammalia, including but not limited to, humans, domestic animals, farm animals, zoo animals, sport animals, pet animals such as dogs, cats, guinea pigs, rabbits, rats, mice, horses, cattle, cows; primates such as apes, monkeys, orangutans, and chimpanzees; canids such as dogs and wolves; felids such as cats, lions, and tigers; equids such as horses, donkeys, and zebras; food animals such as cows, pigs, and sheep; ungulates such as deer and giraffes; rodents such as mice, rats, hamsters and guinea pigs; etc. In certain approaches, the mammal is a human subject. The term does not denote a particular age or sex. Thus, adult and newborn subjects, as well as fetuses, whether male or female, are intended to be included within the scope of this term.

In various approaches, a replacement heart valve is a prosthetic valve or a bioprosthetic valve. In accordance with the present invention, a prosthetic valve is made of purely artificial or non-biological materials, and a bioprosthetic valve is made of animal tissues alone or in combination with artificial or non-biological materials. Materials which may be used to construct a replacement heart valve are well known in the art, for example as described in U.S. Publication No. US2011/0319989, which is incorporated by reference herein in its entirety as fully set forth.

In various approaches, a replacement heart valve is balloon expandable. Examples of balloon-expandable valves include, but are not limited to, Edwards SAPIEN 3 and SAPIEN 3 Ultra Transcatheter Heart Valve (THV), each valve constructed with bovine tissue attached to a balloon-expandable, cobalt-chromium frame for support. Another example of a balloon-expandable valve includes, but is not limited to, Edwards SAPIEN XT VALVE, which is constructed with a cobalt-chromium balloon-expandable valve stent frame and bovine tissue.

In various approaches, the appropriate guide wires, sheaths and catheters for use with the devices, systems and methods described herein will be apparent to a person of skill in the art, for example, as described in Ye et al. (Transapical aortic valve implantation in humans. Ye J, Cheung A, Lichtenstein S V, Carere R G, Thompson C R, Pasupati S, Webb J G. J Thorac Cardiovasc Surg. 2006 May; 131(5):1194-6), Lichtenstein et al. (Transapical transcatheter aortic valve implantation in humans: initial clinical experience. Lichtenstein S V, Cheung A, Ye J, Thompson C R, Carere R G, Pasupati S, Webb J G. Circulation. 2006 Aug. 8; 114 (6):591-6. Epub 2006 Jul. 31), Kurra et al. (Pre-procedural imaging of aortic root orientation and dimensions: comparison between X-ray angiographic planar imaging and 3-dimensional multidetector row computed tomography. Kurra V, Kapadia S R, Tuzcu E M, Halliburton S S, Svensson L, Roselli E E, Schoenhagen P. JACC Cardiovasc Interv. 2010 January; 3 (1):105-13), Wake et al. (Computed tomography angiography for transcatheter aortic valve replacement. Wake N, Kumamaru K, Prior R, Rybicki F J, Steigner M L. Radiol Technol. 2013 March-April; 84(4):326-40), and Little et al. (Multimodality noninvasive imaging for transcatheter aortic valve implantation: a primer. Little S H, Shah D J, Mahmarian J J. Methodist Debakey Cardiovasc J. 2012 April-June; 8(2):29-37), all of which are incorporated by reference herein in their entirety as fully set forth.

According to one general approach, a method is for preparing a prosthetic heart valve for a transcatheter heart valve procedure. The method includes: determining a native heart valve commissural orientation according to one or more images acquired via a cardiac imaging modality. In response to the determination, the prosthetic heart valve is further crimped according to the native heart valve orientation.

According to another general approach, a method is for crimping a prosthetic heart valve. Here, the method includes: determining a crimping orientation according to a native heart valve orientation. The prosthetic heart valve is further positioned in a crimping aperture of a crimping device at the crimping orientation, and a crimping lever is actuated to actually crimp the prosthetic valve.

According to yet another general approach, a method is for crimping a prosthetic heart valve. The method includes: determining at least one native commissure orientation of a native heart valve, and crimping the prosthetic heart valve according to the native commissure orientation.

According to another general approach, a device is for crimping a prosthetic heart valve. The device includes: an external housing including a crimping aperture, and a circular scale disposed around a circumference of the crimping aperture. The circular scale includes radially oriented crimping angles, and the radially oriented crimping angles correlate with one or more native heart commissure angles.

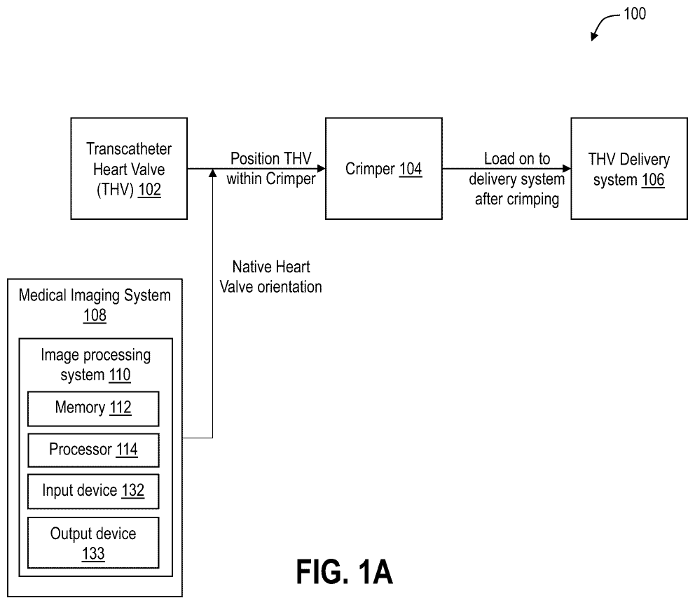

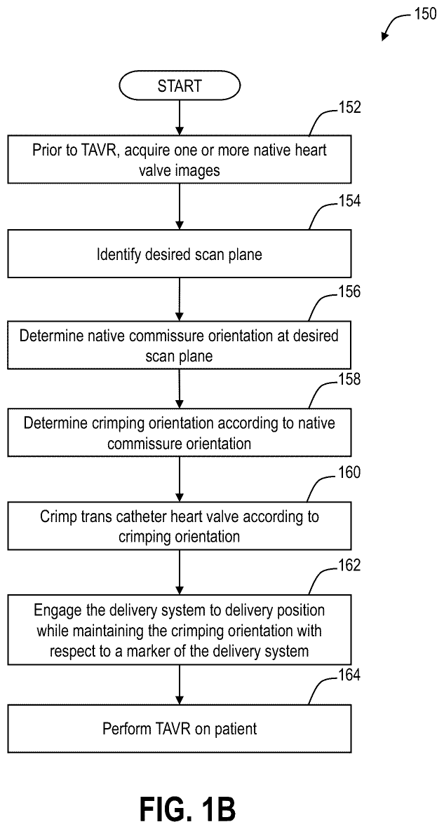

Referring now to FIG. 1A, it shows a block diagram of an example transcatheter heart valve (THV) system 100 that may be utilized for a transcatheter heart valve replacement (THVR) procedure. In particular, the THV system 100 may be utilized for preparing a THV 102 for a THVR procedure in some approaches. As an option, the present THV system 100 may be implemented in conjunction with features from any other approach listed herein, such as those described with reference to the other FIGS. However, such THV system 100 and others presented herein may be used in various applications and/or in permutations which may or may not be specifically described in the illustrative approaches listed herein. Further, the THV system 100 presented herein may be used in any desired environment. Thus FIG. 1A (and the other FIGS.) may be deemed to include any possible permutation.

For instance, the THV system 100 described at FIG. 1A, as well as other systems and methods herein, are described with respect to implanting a balloon-expandable transcatheter aortic valve over a diseased native aortic valve or a degenerated bioprosthetic aortic valve (e.g., such as during a valve-in-valve procedure). However, it will the appreciated that the systems and methods described herein can be implemented for any other transcatheter heart valve implantation procedure, such as a transcatheter mitral valve replacement (TMVR), transcatheter pulmonary valve replacement (TPVR), transcatheter tricuspid valve replacement (TTVR), etc., without departing from the scope of the disclosure. For example, the crimping of the transcatheter heart valve according to the native heart valve orientation (which may be determined using a medical image) described herein can be implemented with respect to any transcatheter heart valve, such as a transcatheter mitral valve, transcatheter pulmonary valve, transcatheter tricuspid valve, etc., without departing from the scope of the disclosure. Further, the crimping of the transcatheter heart valve according to the native heart valve orientation described herein can be implemented with respect to any balloon-expandable transcatheter heart valve, such as a balloon-expandable transcatheter mitral valve, balloon-expandable transcatheter pulmonary valve, balloon-expandable transcatheter tricuspid valve, etc., without departing from the scope of the disclosure. Further, while the crimping technique according to native heart valve orientation is only used for balloon-expandable valves, the method of identifying commissural orientation in the transcatheter valve and advancing the valve delivery system in the body while maintaining the fixed orientation of the valve delivery system can be applied to any transcatheter heart valve that would be apparent to one skilled in the art after reading the present description. It should also be noted that a transcatheter heart valve may be more generally referred to herein as a “prosthetic” heart valve. It follows that in some approaches, the balloon expandable transcatheter heart valve is actually a “prosthetic heart valve.”

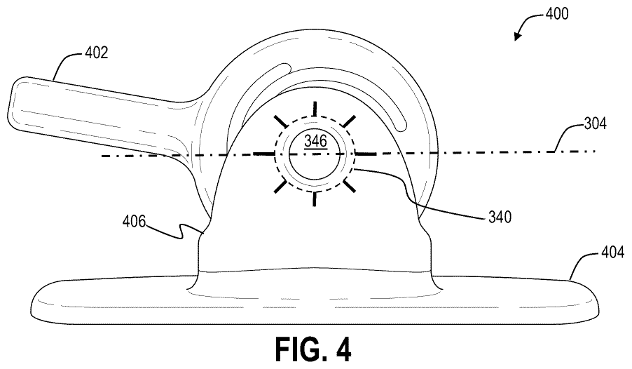

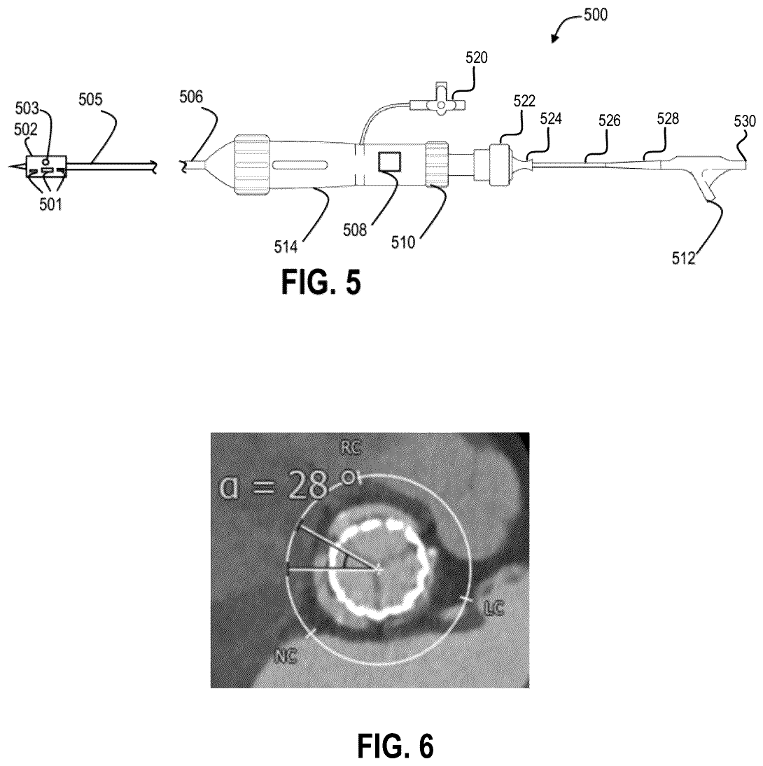

The THV system 100 comprises a THV 102, which may be a transcatheter aortic valve. Further, the THV system 100 comprises a crimper 104 for crimping the THV 102 to reduce a diameter of the THV performing a THVR procedure. For example, a valve crimping section of a delivery system 106 is positioned coaxially with the THV 102, which is positioned within a crimping aperture of the crimper 104 during crimping. An example THV is shown at FIG. 3, an example crimper is shown at FIG. 4, and an example delivery system is shown at FIG. 5.

Further, the THV system 100 includes a medical imaging system 108, such as a computed tomography (CT) system, a cardiac magnetic resonance imaging (MRI) imaging system, a transesophgeal echocardiogram (TEE), etc., or any other cardiac imaging system for acquiring medical images of a native heart. In this example, an image processing system 110 is incorporated into the medical imaging system 108. In some approaches, at least a portion of image processing system 110 is disposed at a device (e.g., edge device, server, etc.) communicably coupled to the medical imaging system via wired and/or wireless connections. In some approaches, at least a portion of the image processing system 110 is disposed at a separate device (e.g., a workstation) which can receive images from the medical imaging system 108, or from a storage device which stores the images generated by the medical imaging system 108.

Further, the medical image processing system 110 may comprise a user input device 132, and display device 133. In some approaches, user input device 132 enables a medical imaging system operator to input/select an imaging protocol. User input device 132 may comprise one or more of a touchscreen, a keyboard, a mouse, a trackpad, a motion sensing camera, or other devices configured to enable a user to interact with, and manipulate data within, image processing system 110. Display device 133 may include one or more display devices utilizing virtually any type of technology. In some approaches, display device 133 may comprise a computer monitor, and may display diagnostic-scan region previews, calibration images, diagnostic images, scan boxes, landmark maps, etc. as part of one or more of the approaches disclosed herein. Display device 133 may be combined with processor 114, non-transitory memory 112, and/or user input device 132 in a shared enclosure, or may be peripheral display devices and may comprise a monitor, touchscreen, projector, or other display device known in the art, which may enable a user to view medical images produced by the medical imaging system 108, and/or interact with various data stored in non-transitory memory 112.

Image processing system 110 includes a processor 114 configured to execute machine readable instructions stored in non-transitory memory 112. Processor 114 may be single core or multi-core, and the programs executed thereon may be configured for parallel and/or distributed processing. In some approaches, the processor 114 may optionally include individual components that are distributed throughout two or more devices, which may be remotely located and/or configured for coordinated processing. In some approaches, one or more aspects of the processor 114 may be virtualized and executed by remotely-accessible networked computing devices configured in a cloud computing configuration. In such approaches, the network may be of any type, e.g., depending on the desired implementation. For instance, an illustrative list of other network types which the network may implement includes, but is not limited to, a wide area network (WAN), local area network (LAN), a public switched telephone network (PSTN), a storage area network (SAN), an internal telephone network, etc. Accordingly, the on-premise processor 114 may be able to communicate with networked computing devices regardless of the amount of separation which exists therebetween, e.g., despite being positioned at different geographical locations.

Non-transitory memory 112 may further store medical image data, which may comprise medical images captured by the medical imaging system 108. In some approaches, non-transitory memory 112 may include components disposed at two or more devices, which may be remotely located and/or configured for coordinated processing. In some approaches, one or more aspects of the non-transitory memory 112 may include remotely-accessible networked storage devices configured in a cloud computing configuration. Moreover, this communication between the non-transitory memory 112 and various locations like a remote networked storage device may be facilitated using any one or more of the network types described above.

It should be understood that the medical image system 108 and the image processing system 110 shown in FIG. 1A is for illustration, and is in no way intended to be limiting. For instance, other appropriate imaging systems and associated image processing systems may include more, fewer, or different components.

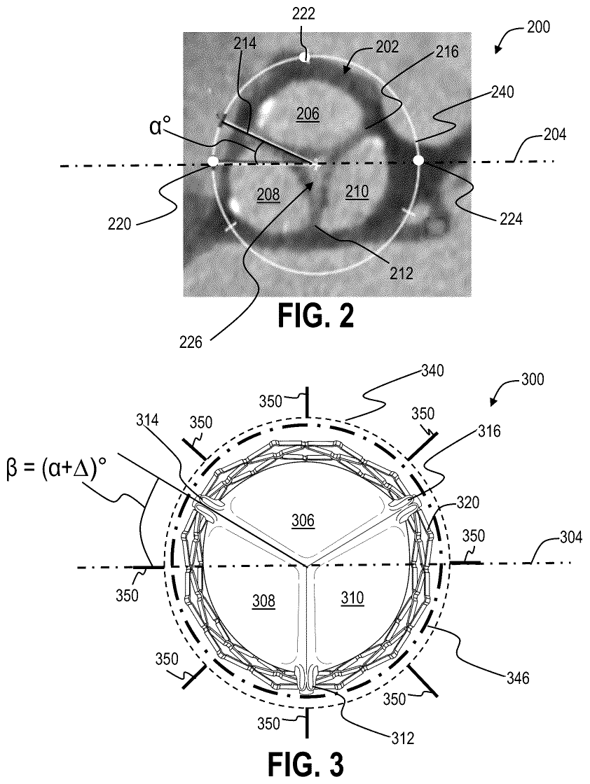

The medical imaging system 108 may be configured to acquire one or more images of the native heart. In one example, the medical imaging system may be a computed tomography (CT) system, and may be configured to acquire one or more CT images of the native heart prior to the TAVR procedure. Using the one or more acquired CT images, a native heart orientation may be determined as further discussed below in further detail with respect to FIGS. 1B, and 2. It will be appreciated that the native heart orientation may be determined based on images acquired from any other cardiac imaging modality such as cardiac MRI, TEE, etc.

The crimper 104 may include one or more crimper indications corresponding to native heart valve orientation. During a crimping procedure, the THV 100 may be positioned within a crimping aperture of the crimper 104 according to the native heart valve orientation. This native heart valve orientation may in turn be determined using one or more images acquired via the medical imaging system 108 using the one or more indications on the crimper. Details of positioning a THV within the crimper and crimping the THV according to the native heart valve orientation is described below in further detail with respect to FIGS. 1B, 2, and 3.

The delivery system 106 may be utilized to load the crimped THV and deliver the THV percutaneously for implantation. In some examples, the delivery system may additionally include one or more delivery system indications corresponding to one or more crimper orientations so as to maintain a crimping orientation of the THV even during delivery and deployment of the THV. As a result, further improvements to the commissural alignment of the THV with the native heart valve may desirably be achieved. An exemplary delivery system is described in further detail below with respect to FIG. 5.

This is particularly desirable, as previous implementations of THVR (and therefore, TAVR) have not taken into consideration the orientation of the native heart valve. As such, when the THV has conventionally been deployed in a patient by using a THVR procedure, commissure alignment is not achieved in a majority of patients, which causes issues in terms of coronary access, valve-in-valve replacement, etc. Again, the inventors have identified a method for achieving increased commissure alignment between THV and native heart valves in patients undergoing THVR, e.g., as will be described in further detail below.

Next, FIG. 1B shows a flow chart illustrating a high-level method 150 for performing a transcatheter heart valve replacement procedure, in accordance with an embodiment. The method 150 will be described with respect to FIGS. 2-5, although it will be appreciated that the method 150 may be implemented for any transcatheter heart valve replacement procedure without departing from the scope of the disclosure. The method 150 may be performed in accordance with the present invention in any of the environments associated with FIG. 1A, among others, in various approaches. Of course, more or less operations than those specifically described in FIG. 1B may be included in method 150, as would be understood by one of skill in the art after reading the present description.

Each of the steps of the method 150 may be performed by any suitable component of the operating environment. For example, in various approaches, the method 150 may be partially or entirely performed by a controller, a processor (e.g., see 114 of FIG. 1A), a computer, etc., or some other device having one or more processors therein. Thus, in some approaches, method 150 may be a computer-implemented method. Moreover, the terms computer, processor and controller may be used interchangeably with regards to any of the approaches herein, such components being considered equivalents in the many various permutations of the present invention.

Moreover, for those approaches having a processor, the processor, e.g., processing circuit(s), chip(s), and/or module(s) implemented in hardware and/or software, and preferably having at least one hardware component, may be utilized in any device to perform one or more steps of the method 150. Illustrative processors include, but are not limited to, a central processing unit (CPU), an application specific integrated circuit (ASIC), a field programmable gate array (FPGA), etc., combinations thereof, or any other suitable computing device known in the art.

As shown at 152, method 150 includes, acquiring one or more native heart valve images via a medical imaging system prior to performing a transcatheter heart valve replacement (THVR) procedure on a patient. According to some approaches, the one or more native heart valve images may be acquired using the medical imaging system 108 described at FIG. 1A. In one example, the THVR procedure may be a transcatheter aortic valve replacement (TAVR) procedure, and accordingly, a native aortic valve complex of the patient may be imaged. In various instances, the native aortic valve complex may be imaged with a CT, an ultrasound (intravascular or echocardiography), an MRI imaging system, etc. In some approaches, a native mitral valve complex, a native pulmonary valve complex, a native tricuspid valve complex, etc., may additionally or alternatively be imaged, e.g., depending on the type of procedure.

Next, at 154, the method 150 includes identifying a desired scan plane. In one example, the desired scan plane is a plane showing a cross-sectional image of the native heart valve. Accordingly, when a TAVR procedure is performed, the desired scan plane is a plane substantially perpendicular to an axis of the aorta, the plane showing a cross-sectional image of the native aortic valve. In particular, the cross-sectional image may be identified based on aortic valve leaflet coaptation where the three leaflets of the native aortic valve (that is, the left coronary leaflet, the right coronary leaflet, and the non-coronary leaflet of the native aortic valve) meet in a triangular formation. That is, the desired scan plane shows a cross-sectional image of the native heart valve perpendicular to an aorta axis and showing a triangular formation of leaflet coaptation. An example cross-sectional image of the native aortic valve, which is in no way intended to be limiting, is shown at FIG. 2.

Next, at 156, the method 150 includes determining a native heart valve orientation at the desired scan plane. In particular, determining the native heart valve orientation involves determining a native commissural orientation at the desired scan plane in preferred approaches. Determining the native commissural orientation may include determining an angular position of a first commissure between a non-coronary leaflet and a right-coronary leaflet of the native aortic valve at the desired scan plane. The angular position of the first commissure may further be determined with respect to a reference axis passing through the triangular formation. Said another way, determining the native commissure orientation may include determining a native commissure angle with respect to a reference axis, the reference axis passing through a central area of meeting of a right-coronary leaflet, a left-coronary leaflet, and a non-coronary leaflet, e.g., as will soon become apparent.

The determination of the native commissural orientation is described with respect to FIG. 2 below, which again is in no way intended to be limiting.

Referring now to FIG. 2, it shows a cross-sectional CT image 200 (that is, image at the desired scan plane) of a native aortic heart valve 202 having a right-coronary leaflet 206, a non-coronary leaflet 208, and a left coronary leaflet 210. Further, a leaflet coaptation is shown by a central triangular formation 226 (also referred to as central area of meeting of the right-coronary, left-coronary, and non-coronary leaflets). With an outer arc edge of the right-coronary leaflet 206 having a mid-point at a twelve o'clock position (222), a reference axis 204 is set that passes through a three o'clock position (220), a center of the triangular formation 226, and a nine o'clock position (224) of a reference clock-face circle shown in white drawn around the cross-section of the native heart valve, the circle having a center at the center of the triangular formation. An angular position of a first commissure 214 is indicated by α°. In this example a may be about 23°. The angular position of the first commissure 214 between the right-coronary leaflet 206 and the non-coronary leaflet 208 provides an indication of the native commissure orientation. Additionally, or alternatively, one or more of the angular positions of a second commissure 216 between the right-coronary leaflet 206 and the left-coronary leaflet 210, and/or a third commissure 212 between the left-coronary leaflet 210 and the non-coronary leaflet 208, may be used to determine and/or evaluate the native commissure orientation, and thus, the native heart orientation.

In this way, a native heart valve orientation may be determined based on angular orientations of one or more commissures of the native heart valve. In patients with bicuspid aortic valve undergoing TAVR, position of any of the two commissures may be considered with respect to the reference axis for native heart valve orientation.

In one example, the steps 152, 154, and 156 described above for determining native heart valve orientation may be performed by a processor, such as processor 114 at FIG. 1A, according to executable instructions stored in non-transitory memory, such as memory 112 at FIG. 1A, of a medical imaging system acquiring the one or more medical images. For example, in response to acquiring one or more images of the heart, an image processing system may automatically identify a desired cross-sectional image and determine a native heart valve orientation according to orientations of one or more commissures.

Returning to FIG. 1B, in response to determining the native heart valve orientation according to the native commissure orientation at step 156, the method 150 proceeds to 158. At 158, the method 150 includes determining a crimping orientation according to the native commissure orientation.

Previous methods for THVR (and therefore, TAVR) do not take in to consideration orientation of the native heart valve. As such, conventionally when a THV is deployed in a patient by using a THVR procedure, commissure alignment has not been achieved in a majority of patients, which has caused issues in terms of coronary access, valve-in-valve replacement, etc.

In response to these conventional shortcomings, the inventors have identified a method for achieving increased commissure alignment between THV and native heart valves in patients undergoing THVR. Accordingly, a method for preparing a THV for THVR according to various ones of the approaches included herein may include determining a native heart valve orientation and crimping the THV according to the native heart valve orientation. In particular, this method may include crimping the THV according to native commissure orientation. By taking into account the native commissure orientation, and crimping the THV based on the native heart valve commissure orientation, increased commissure alignment is achieved, which has significantly improved patient outcomes after THVR. Thus, a crimping orientation is preferably based on the native heart commissure orientation. In one example, orientation of the commissure between the right-coronary leaflet and the non-coronary leaflet is used for determining the native heart valve commissure orientation. However, orientation of any commissure of the native heart valve may be used determining crimping orientation, e.g., depending on the particular approach.

Crimping orientation β is an angular position of a commissure post of the THV. The crimping orientation is a function of angular position of a commissure of the native heart valve. In one example, angular position of the commissure post of the THV is a function of the angular position of the first commissure (e.g., the first commissure 214) between the right-coronary leaflet and the non-coronary leaflet. In some other examples, the angular position of the commissure post of the THV may be a function of an angular position of the second commissure (e.g., second commissure 216) between the right coronary leaflet and the left coronary leaflet, and/or an angular position of the third commissure (e.g., third commissure 212) between the left coronary leaflet and the non-coronary leaflet.

Looking now to FIG. 3, an exemplary crimping orientation of a transcatheter heart valve 300 is shown and described with respect to the elements illustrated in FIG. 2 and in the corresponding description above. However, it should be noted that, as an option, the present crimping orientation may be implemented in conjunction with features from any other approach listed herein, such as those described with reference to the other FIGS. However, such crimping orientation and others presented herein may be used in various applications and/or in permutations which may or may not be specifically described in the illustrative approaches listed herein. Further, the crimping orientation presented herein may be used in any desired environment.

As shown, FIG. 3 illustrates a transcatheter heart valve 300, which includes a frame 320 in mechanical contact with the valve tissue (i.e., leaflets 306, 308 and 310), at a crimping orientation β and with respect to a crimper aperture 346 and a circular scale 340 of a crimper. The crimping aperture 346 is shown in thicker dash-dot-dash lines, and the circular scale 340 is shown in dashed lines. In this example, the THV 300 is oriented based at least in part on the native heart valve orientation α, e.g., shown at FIG. 2 and shown in the crimper aperture 346 of a crimper 400 shown at FIG. 4. During crimping, the THV 300 is positioned in the crimping aperture 346 of the crimper 400 at the crimping orientation β. In this example, the crimping orientation is an angular position of a commissure post 314 (between first leaflet 306 and second leaflet 308) with respect to reference axis 304. Accordingly, the crimping orientation β=α+Δ, where α is the angular position of the first commissure 214 of the native aortic heart valve 202, and Δ is an adjustment angle to account for rotation of THV during delivery and deployment within a patient. While the above example describes setting a crimping orientation using the commissure post 314, other commissure posts 316 and/or 312 may be used.

In one example, the crimping orientation is directly proportional to native commissure orientation. For example, a native commissure orientation of a first commissure (the first commissure between a right-coronary leaflet and a non-coronary leaflet) increases with respect to a reference axis (e.g., reference axis 204). A position of a commissure of the THV with respect to a corresponding reference axis of a crimper may also increase.

Returning now to FIG. 1B, in response to determining the crimping orientation, at 160, the method 150 includes positioning the THV in the crimper and actuating a crimper handle (e.g., “lever”) to crimp the THV at the crimping orientation. It should be noted that with respect to the present description, a crimper handle (or crimper lever) is intended to refer to a mechanical sub-system that can be engaged by a user (e.g., human) in order to actuate the crimper 400. Positioning the THV within the crimper desirably includes positioning a valve crimp section of a delivery system coaxially with the THV, as well as positioning the THV and the valve crimp section within a crimping aperture of the crimper. Further, in some examples, one or more crimping accessories may be included in order to crimp the THV to a desired diameter. An exemplary delivery system that may be used during the crimping process is shown and described below with respect to FIG. 5.

Looking now to FIG. 4, a crimper 400 is shown in accordance with one embodiment. As an option, the present crimper 400 may be implemented in conjunction with features from any other approach listed herein, such as those described with reference to the other FIGS., such as FIG. 1B. However, such crimper 400 and others presented herein may be used in various applications and/or in permutations which may or may not be specifically described in the illustrative approaches listed herein. Further, the crimper 400 presented herein may be used in any desired environment. Thus FIG. 4 (and the other FIGS.) may be deemed to include any possible permutation.

Turning to FIG. 4, the crimper 400 includes a crimper handle 402 and base 404. Further, crimper 400 includes crimping aperture 346 in which THV 300 is positioned for crimping. Further, crimper 400 includes one or more indications corresponding to a native heart valve orientation. In one example, e.g., as shown at FIGS. 3 and 4, the indications may be a circular scale 340 having radially orientated indications 350 corresponding to a plurality of angles. Moreover, the circular scale may be disposed on an external housing 406 of the crimping device 400. In other words, in some approaches the circular scale may be disposed around a circumference or outermost rim of the crimping aperture, the circular scale including radially oriented crimping angles.

A circular scale 346 having radially oriented indications 350 represents a crimping orientation that corresponds to native heart orientation. This also desirably provides the functionality of positioning a THV in the crimper corresponding to the native heart valve orientations. That is, the indications 350 on the circular scale 346 provide a commissure post angular position of a commissure post that may be used during crimping. The circular scale may range from 0 to 360 degrees, with indications provided at any desired degree intervals. In various approaches, the desired degree intervals may be 1, 2, 3, 4, 5, and so on up to 45 degrees, but the intervals may be any size, at any regular and/or changing frequency, etc. In one example, the crimping orientation angle (that is, β=α+Δ) may be calculated based on the native heart valve commissure angle (that is, α) and the THV may be positioned at the crimping orientation angle β based on indications 350 that are on the circular scale 346. In another example, the crimping orientation angle may be the same as the native heart valve commissure angle, and as such the THV may be positioned at the crimping orientation angle β which may be the same as the native heart valve commissure angle α.

In one example, two circular scales positioned in an annular manner may be provided. Each of the circular scales may further correspond to the orientation (e.g., angular position) of pertinent biological and/or inorganic components. For example, a first circular scale provides indications corresponding to the native heart valve angle, and a second circular scale provides indication corresponding to an adjustment angle added to the native heart valve angle.

In another example, a locking mechanism may be included in the crimper so that the transcatheter heart valve does not rotate while being crimped on to the delivery system. For example, once the THV is positioned within the crimper at the desired crimping orientation, the locking mechanism may maintain the position of the THV at the desired crimping orientation such that the rotation of THV is reduced or minimized during crimping.

In other examples, one or more additional indications may be provided on the crimper in additional to the circular scale(s), where the one or more additional indications provide a desired position of an element of a THV delivery system, e.g., such as a THV delivery system shown at FIG. 5. Turning to FIG. 5, an exemplary THV delivery system 500 is shown in accordance with one embodiment. As an option, the present THV delivery system 500 may be implemented in conjunction with features from any other approach listed herein, such as those described with reference to the other FIGS. However, such THV delivery system 500 and others presented herein may be used in various applications and/or in permutations which may or may not be specifically described in the illustrative approaches listed herein. Further, the THV delivery system 500 presented herein may be used in any desired environment. Thus FIG. 5 (and the other FIGS.) may be deemed to include any possible permutation.

As shown, the THV delivery system 500 includes a valve crimp section 505. The valve crimp section is positioned coaxially within the THV and crimped during a crimping of the THV valve. Proximal to the valve crimp section 505 (that is, towards a tapering tip of the delivery system at an end opposite to a balloon inflation port 512), the delivery system 500 includes a valve alignment section 502. In some approaches, the valve alignment section may include a commissure orientation marker 503 in addition to one or more valve alignment markers 501. The delivery system 500 also includes a flush port 520 that may be used to flush (e.g., with a liquid, air, solvent, etc.) any contents from the system, in addition to: a balloon lock 522, strain relief 524 that may work in conjunction with the balloon inflation port 512, a balloon catheter 526, volume indicator 528, and guidewire lumen, each or some of which may be used to selectively adjust and lock a desired amount of pressure. As a result, the approach is desirably able to maintain the position of the balloon inflation port 512 while advancing the valve, e.g., as would be appreciated by one skilled in the art after reading the present description.

Further, the crimping orientation marker on the crimping device is positioned at a pre-determined angle on the clock face. Then, the commissure post of the THV is positioned such that the commissure post is aligned with the crimping orientation marker on the crimping device. In an example, when coaxially positioned, the valve crimp section may be oriented such that the commissure orientation marker 503 is positioned at the native heart valve orientation angle (that is native commissure angle α) or adjusted angle β, where β=α+Δ, as discussed above. In particular, the valve crimp section of the delivery system is positioned in the crimper, at angle α or β, relative to the E-marker on the valve delivery system or any other constant marker that can be identified on the delivery system. Although not depicted, the valve crimp section 505 may also include a triple marker which may be used in some procedures, e.g., as would be appreciated by one skilled in the art after reading the present description. The THV delivery system 500 also includes a flex catheter 506 that effectively couples the valve crimp section 505 to a body of the THV delivery system 500, e.g., as shown.

Returning again to FIG. 1B, in response to performing the crimping, the method 150 proceeds from 160 to 162. At 162, the method 150 includes engaging the delivery system to a delivery position while maintaining the crimped valve at the crimping orientation with respect to a crimping orientation marker of the delivery system. In one example, the crimping orientation marker is the commissure orientation marker 503 of the delivery system. In other examples, the crimping orientation marker may be a marker 508 on a body 514 of the delivery system 500. However, it should be noted that any other desired marker may be used, e.g., such as balloon inflation port 512, a flush port (not shown), and/or fine adjustment wheel 510 of the delivery system 500.

Next, at 164, the method 150 includes performing TAVR on the patient. In some approaches one or more positions of the one or more marker (503, or 508, or port 512) may be maintained during delivery and deployment for commissural alignment.

Although not depicted in the high-level method 150 of FIG. 1B, in various approaches, the method may further include inserting an instrument into the central lumen and advancing the instrument through the aortic valve orifice into the left ventricle. Examples of the instrument that may be used include, but are in no way limited to, a: tube, sheath, guidewire, catheter, balloon, stent, needle, pressure sensor, etc. It follows that in some approaches, method 150 of FIG. 1B may additionally include inserting a TAVR delivery device into the central lumen and delivering a TAVR device (that is, transcatheter aortic valve) to the aortic valve.

An example commissural alignment of a THV with a native heart valve is shown by a medical image 600 at FIG. 6 which is in no way intended to be limiting. As discussed above, an orientation of the native aortic commissures is determined based on the pre-TAVR imaging with CT. Further, the orientation of the native aortic valve commissures is also confirmed with transesophageal echocardiogram (TEE) that is routinely performed during the TAVR procedure.

Image 600 shows the orientation of the THV commissures after valve deployment with TEE imaging. As shown, an angular position of a commissure post is 28 degrees. That is, the angular position of a commissure post of the THV (the commissure post between a THV right-coronary leaflet (RC) and a THV non-coronary leaflet (NC)) with respect to a reference line passing through the center of the valve is 28 degrees.

This angular value may further be determined as being within a predetermined threshold deviation range from an angular position of a commissure (which is 23 degrees in this example) of the native heart valve between a native right-coronary leaflet and a native non-coronary leaflet. According to an example, which is in no way intended to be limiting, the angular value may be compared against a predetermined threshold deviation range of about 15 degrees. In other words, tolerance may allow for the actual angular value to be ±7.5 degrees from an intended (e.g., ideal) angular value. Furthermore, the range may be predetermined by a user, a medical professional, a system administrator, based on industry standards, etc. In other approaches, the range may actually adjust dynamically based on real-time factors such as patient specific information, past procedure outcomes, a medical professional's assessment of the situation, etc.

Also in accordance with the present invention, as TAVR is a known surgical procedure, one of ordinary skill in the art would readily recognize that the method could involve other additional steps, which are not described in details here. These additional steps include, but are not limited to, anesthesia, sterilization, heparinization, accessing the patient's heart via various routes such as femoral, transseptal, transaortic and transapical approaches; ventricular pacing, stitching of the access site, percutaneous femoral closure, etc. For example, more information on these procedures are described in Ye et al. (Transapical aortic valve implantation in humans. Ye J, Cheung A, Lichtenstein S V, Carere R G, Thompson C R, Pasupati S, Webb J G. J Thorac Cardiovasc Surg. 2006 May; 131(5):1194-6), Lichtenstein et al. (Transapical transcatheter aortic valve implantation in humans: initial clinical experience. Lichtenstein S V, Cheung A, Ye J, Thompson C R, Carere R G, Pasupati S, Webb J G. Circulation. 2006 Aug. 8; 114 (6):591-6. Epub 2006 Jul. 31), Kurra et al. (Pre-procedural imaging of aortic root orientation and dimensions: comparison between X-ray angiographic planar imaging and 3-dimensional multidetector row computed tomography. Kurra V, Kapadia S R, Tuzcu E M, Halliburton S S, Svensson L, Roselli E E, Schoenhagen P. JACC Cardiovasc Interv. 2010 January; 3 (1):105-13), Wake et al. (Computed tomography angiography for transcatheter aortic valve replacement. Wake N, Kumamaru K, Prior R, Rybicki F J, Steigner M L. Radiol Technol. 2013 March-April; 84(4):326-40), and Little et al. (Multimodality noninvasive imaging for transcatheter aortic valve implantation: a primer. Little S H, Shah D J, Mahmarian J J. Methodist Debakey Cardiovasc J. 2012 April-June; 8(2):29-37), all of which are incorporated by reference herein in their entirety as fully set forth.

The various methods and techniques described above provide a number of ways to carry out the application. Of course, it is to be understood that not necessarily all objectives or advantages described can be achieved in accordance with any particular embodiment and/or approach described herein. Thus, for example, those skilled in the art will recognize that the methods can be performed in a manner that achieves or optimizes one advantage or group of advantages as taught herein without necessarily achieving other objectives or advantages as taught or suggested herein. A variety of alternatives are mentioned herein. It is to be understood that some preferred embodiments and/or approaches specifically include one, another, or several features, while others specifically exclude one, another, or several features, while still others mitigate a particular feature by inclusion of one, another, or several advantageous features.

Furthermore, the skilled artisan will recognize the applicability of various features from different embodiments and/or approaches. Similarly, the various elements, features and steps discussed above, as well as other known equivalents for each such element, feature or step, can be employed in various combinations by one of ordinary skill in this art to perform methods in accordance with the principles described herein. Among the various elements, features, and steps some will be specifically included and others specifically excluded in diverse embodiments and/or approaches.

Although the application has been disclosed in the context of certain embodiments, approaches, examples, etc., it will be understood by those skilled in the art that the description of the application extend beyond the specifically disclosed embodiments, approaches, examples, etc., to other alternative implementations and/or uses and modifications and equivalents thereof.

In some embodiments and/or approaches, the terms “a” and “an” and “the” and similar references used in the context of describing a particular aspect of the application (especially in the context of certain of the following claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (for example, “such as”) provided with respect to certain embodiments and/or approaches herein is intended merely to better illuminate the application and does not pose a limitation on the scope of the application otherwise claimed. No language in the specification should be construed as indicating any non-claimed element essential to the practice of the application.

Preferred embodiments and/or approaches of this application are described herein, including the best mode known to the inventors for carrying out the application. Variations on those preferred embodiments and/or approaches will become apparent to those of ordinary skill in the art after reading the foregoing description. It is contemplated that skilled artisans can employ such variations as appropriate, and the application can be practiced otherwise than specifically described herein. Accordingly, many embodiments and/or approaches of this application include all modifications and equivalents of the subject matter recited in the claims appended hereto as permitted by applicable law. Moreover, any combination of the above-described elements in all possible variations thereof is encompassed by the application unless otherwise indicated herein or otherwise clearly contradicted by context.

All patents, patent applications, publications of patent applications, and other material, such as articles, books, specifications, publications, documents, things, and/or the like, referenced herein are hereby incorporated herein by this reference in their entirety for all purposes, excepting any prosecution file history associated with same, any of same that is inconsistent with or in conflict with the present document, or any of same that may have a limiting affect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

It is to be understood that the embodiments, approaches, examples, etc. of the application disclosed herein are illustrative of the principles of the embodiments, approaches, examples, etc. of the application. Other modifications that can be employed can be within the scope of the application. Thus, by way of example, but not of limitation, alternative configurations of the embodiments, approaches, examples, etc. of the application can be utilized in accordance with the teachings herein. Accordingly, embodiments, approaches, examples, etc. of the present application are not limited to that precisely as shown and described.

What is claimed is:

1. A method for preparing a prosthetic heart valve for a transcatheter heart valve procedure, the method comprising:
determining a native heart valve commissural orientation according to one or more images acquired via a cardiac imaging modality; and
crimping the prosthetic heart valve according to the native heart valve orientation.

2. The method of claim 1, wherein determining the native heart valve orientation includes determining a native commissure orientation according to the one or more images.

3. The method of claim 2, wherein determining the native commissure orientation includes identifying a desired scan plane in the one or more images, wherein the one or more images are of a native heart of a patient undergoing a valve replacement procedure with the crimped prosthetic heart valve.

4. The method of claim 3, wherein the desired scan plane is a cross-sectional view plane of the native heart valve.

5. The method of claim 3, wherein determining the native commissure orientation further includes determining a native commissure angle with respect to a reference axis, the reference axis passing through a central area of meeting of a right-coronary leaflet, a left-coronary leaflet, and a non-coronary leaflet.

6. The method of claim 5, wherein the native commissure angle is an angle of a first commissure between the non-coronary leaflet and the right coronary leaflet or a second angle of a second commissure or a third angle of a third commissure.

7. The method of claim 2, wherein crimping the prosthetic heart valve according to the native heart valve orientation includes:
determining a crimping orientation according to the native commissure orientation; and
positioning a commissure of the prosthetic valve within a crimping device according to the crimping orientation.

8. The method of claim 7, wherein determining the crimping orientation includes:
determining a correction factor, the correction factor based on an expected change in orientation of the prosthetic valve during deployment of the prosthetic valve.

9. The method of claim 7, wherein a position of the commissure at the crimping orientation is with respect to a marker of a delivery system configured for deploying the prosthetic valve.

10. The method of claim 9, wherein the marker is selected from the group consisting of: a balloon inflation port, a flush port, and an indication on the delivery system.

11. The method of claim 1, wherein the prosthetic heart valve is a balloon expandable transcatheter heart valve.

12. The method of claim 1, wherein the cardiac imaging modality is selected from the group consisting of: a computed tomography modality, a magnetic resonance imaging modality, and a transoesophageal echocardiogram modality.

13. A method for crimping a prosthetic heart valve, comprising:
determining a crimping orientation according to a native heart valve orientation;
positioning the prosthetic heart valve in a crimping aperture of a crimping device at the crimping orientation; and
actuating a crimping lever to crimp the prosthetic valve.

14. The method of claim 13, wherein the crimping device includes a circular scale having radially orientated indications corresponding to a plurality of crimping orientations, the circular scale disposed on an external housing of the crimping device.

15. The method of claim 14, wherein the crimping aperture is surrounded by the circular scale.

16. The method of claim 13, wherein determining the crimping orientation according to the native heart orientation includes:
determining a correction factor, the correction factor based on an expected valve position change during deployment.

17. The method of claim 13, wherein determining the crimping orientation according to the native heart orientation includes:
acquiring native heart imaging data via a cardiac imaging modality.

18. The method of claim 17, wherein determining the crimping orientation according to the native heart orientation includes:
selecting a desired cross-sectional image based on the native heart imaging data; and
determining, according to the desired cross-sectional image, one or more of: a first native commissure angle, a second native commissure angle, and a third native commissure angle.

19. The method of claim 18, wherein the one or more of the first, second, and third native commissure angles are determined with respect to a reference axis on the desired cross-sectional image, the reference axis passing through a central portion where a right-coronary leaflet, a left-coronary leaflet, and a non-coronary leaflet of a tricuspid valve meet.

20. The method of claim 13, wherein the prosthetic heart valve is a balloon-expandable heart valve.

21. The method of claim 13, wherein the cardiac imaging modality is selected from the group consisting of: a computed tomography modality, a magnetic resonance imaging modality, and a transoesophageal echocardiogram modality.

22. A method for crimping a prosthetic heart valve, the method comprising:
determining at least one native commissure orientation of a native heart valve; and
crimping the prosthetic heart valve according to the native commissure orientation.

23. The method of claim 22, wherein the at least one native commissure orientation is determined according to at least one image of a native heart of a patient undergoing a valve replacement procedure with the prosthetic heart valve, the at least one image acquired via a cardiac imaging modality.

24. The method of claim 23, wherein the at least one image is a cross-section of the native heart valve, the cross-section being oriented perpendicular to a vertical axis of an aorta of the native heart.

25. The method of claim 23, wherein the at least one image shows the native heart valve in a closed position.

26. The method of claim 22, wherein the at least one native commissure orientation is based on a first commissure between a non-coronary leaflet and a right coronary leaflet of the native heart.

27. The method of claim 23, wherein the valve replacement procedure is a transcatheter valve replacement procedure.

28. The method of claim 22, wherein the prosthetic heart valve is a balloon expandable valve.

29. The method of claim 22, wherein crimping the prosthetic heart valve is performed via a crimping device including a plurality of indications corresponding to a plurality of native commissure orientations, the plurality of indications provided on a circular scale surrounding a rim of a crimping aperture of the crimping device.

30. The method of claim 29, wherein at least one prosthetic commissure of the prosthetic heart valve is positioned in the crimping aperture according to the at least one native commissure orientation.

31. The method of claim 23, comprising:
determining a crimping orientation according to at least one native commissure orientation, the crimping orientation including a correction factor based on expected rotation of the prosthetic heart valve during deployment.

32. A device for crimping a prosthetic heart valve, comprising:
an external housing including a crimping aperture; and
a circular scale disposed around a circumference of the crimping aperture, the circular scale including radially oriented crimping angles,
wherein the radially oriented crimping angles correlate with one or more native heart commissure angles.
